(19) United States
(12) Patent Application Publication
(10) Pub. No.: US 2009/0093739 A1
(43) Pub. Date: Apr. 9, 2009

(54) APPARATUS FOR GENERATING ELECTRICAL DISCHARGES

(76) Inventor: Axel Voss, Kreuzlingen (CH)

Correspondence Address: DAVID L. KING, SR., 5131 N.E. COUNTY ROAD 340, HIGH SPRINGS, FL, 32643, US

(21) Appl. No.: 12/241,232

(22) Filed: Sep. 30, 2008

Related U.S. Application Data
(60) Provisional application No. 60/977,691, filed on Oct. 5, 2007.

(51) Int. Cl. A61B 17/22

(52) U.S. Cl. 601/4

(57) ABSTRACT

The invention relates to an apparatus (2) for generating electrical discharge in a fluid medium (3) in order to generate electrohydraulic shock waves. The electrodes (1) consist of a metallic work material. An electrical voltage is applied to the electrodes (1) in order to generate a voltage breakdown between the tips of the electrodes in the fluid medium (3), which work material consists of a tantalum alloy having a tantalum component greater than 60% or a tungsten alloy having a tungsten component greater than 60%.

### RELATED APPLICATIONS

This application claims priority to provisional application No. 60/977,691 filed Oct. 5, 2007 entitled “Apparatus for Generating Electrical Discharge”.

### TECHNICAL FIELD

The invention relates to an apparatus for generating electrical discharge in a fluid medium in order to generate electrohydraulic shock waves. The apparatus comprises electrodes consisting of a metallic work material. An electrical voltage is applied to the electrodes in order to generate a voltage breakdown between the tips of the electrodes in the fluid medium.

### BACKGROUND OF THE INVENTION

Shock wave generators are used in numerous medical fields.

The best-known field is the therapeutic and cosmetic application in the treatment for instance of calculous diseases (e.g. urolithiasis, cholelithiasis) and the treatment of scars in human and veterinary medicine.

New fields of application relate to dental treatment, the treatment of arthrosis, the 15 ablation of calcium deposits (e.g. tendinosis calcarea), the treatment of chronic tennis or golfer elbows (so-called radial or ulnar epicondylopathy), of chronic discomfort of the shoulder tendons (so-called tendinosis of the rotator cuff), and of chronic irritation of the Achilles tendon (so-called achillodynia).

Furthermore, the generation of shock waves is used in the therapy of osteoporosis, non-healing bone fractures (so-called pseudoarthrosis), bone necroses, and similar diseases. Newer studies also investigate the application in stem cell therapy.

Furthermore, the generation of shock waves can be used to exert mechanical stress, e.g. in the form of shearing forces, on cells, during which their apoptosis is initiated. This happens for example by means of an initiation of the ‘death receptor pathway’ and/or the cytochrome c-pathway and/or a caspase cascade.

The term apoptosis is understood to refer to the initiation of a genetically controlled program which leads to the ‘cell suicide’ of individual cells in the tissue structure. As a result, the cells concerned and their organelles shrink and disintegrate into fragments, the so-called apoptotic bodies. These are phagocytized afterwards by macrophages and/or adjoining cells. Consequently, the apoptosis constitutes a non-necrotic cell death without inflammatory reaction.

Therefore, the application of shock waves is beneficial in all cases, where it relates 5 to the treatment of diseases with a lowered rate of apoptosis, e.g. treatment of tumors or viral diseases.

Additionally, the generation of shock waves can be applied especially beneficially in the treatment of necrotically changed areas and structures in muscle tissue, especially in tissue of the cardiac muscle, in the stimulation of cartilage build-up in 10 arthritic joint diseases, in the initiation of the differentiation of embryonic or adult stem cells in vivo and in vitro in relation to the surrounding cell structure, in the treatment of tissue weakness, especially of cellulitis, and in the degradation of adipose cells, as well as for the activation of growth factors, especially TGF-[beta].

Likewise, the generation of shock waves can be used for avoiding the formation 15 and/or extension of edema, for the degradation of edema, for the treatment of ischemia, rheumatism, diseases of joints, jaw bone (periodontosis), cardiologic diseases and myocardial infarcts, pareses (paralyses), neuritis, paraplegia, arthrosis, arthritis, for the prophylaxis of scar formation, for the treatment of scar formation respectively nerve scarring, for the treatment of achillobursitis and other bone necroses.

Another application relates to the treatment of spinal cord and nerve lesions, for example, spinal cord lesions accompanied by the formation of edema.

Shock waves are also suitable for the treatment of scarred tendon and ligament tissue as well as of poorly healing open wounds.

Such poorly healing open wounds and boils are called ulcus or also ulceration. They are a destruction of the surface by tissue disintegration at the dermis and/or mucosa. Depending on what tissue parts are affected, superficial lesions are called exfoliation (only epidermis affected) or excoriation (epidermis and corium affected).

Open wounds that can be treated with shock waves comprise especially leg ulcers, hypertensive ulcers, varicose ulcers or terebrant ulcers on account of the resulting improved healing process.

Furthermore, shock waves are suitable for the stimulation of cell proliferation and the differentiation of stem cells.

In order to generate shock waves, metallic electrodes are used between which a voltage breakdown takes place by the application of an electrical voltage. The voltage breakdown causes a discharge that for its part generates a short, intensive shock-like pressure wave, a shock wave, in a fluid medium, e.g., water. The shock wave causes a tensile stress in its fluid effective range that produces cavitation bubbles in a regular, chaotic manner that then collapse. If the collapse of the cavitation bubbles takes place in the immediate vicinity of a solid body, this can tear out components of the body, which is desired in the case of a kidney stone. However, the destructive action of the cavitation bubbles also affects the metallic electrodes that are necessary for generating the shock waves.

In this connection the material hardness and the strength of the metallic work material from which the electrodes are manufactured, become more important. However, the harder the work material is and the greater the material strength is, the more difficult it also is to work the material for the manufacture of electrodes. Because the electrodes are used in a fluid medium, the corrosion qualities of the material must also be considered. In addition to the strength features of the work material even the electrical qualities of the work material such as, e.g., the conductivity must also be pointed out here as a selection criterion of the work material. Since the electrodes are used in surgical instruments, they should also consist of a light material to the extent possible. Furthermore, the electrical voltage applied to the electrodes generates a high thermal load for the electrodes.

Therefore, it is desirable that a very strong material with high conductivity, good corrosion resistance, high thermal resistance and a low specific density is made available that can be readily worked.

A material is known from patent U.S. Pat. No. 6,972,116 that is used for the manufacture of electrodes for an apparatus for generating electrohydraulic shock waves. This concerns here a non-ferrous alloy with components of cobalt, nickel and titanium. This alloy has a high thermal loading capacity and a good mechanical workability quality. The specific density of the electrodes is too high on account of the alloy components, cobalt and nickel, which constitutes a weight problem.

The present invention therefore has the task of making an apparatus available for generating electrical discharges in a fluid medium for generating electrohydraulic shock waves, whose electrodes comprise a very strong material with high conductivity, good corrosion resistance, high thermal resistance and a good mechanical workability quality that has a low specific density.

### SUMMARY OF THE INVENTION

The invention relates to an apparatus (2) for generating electrical discharge in a fluid medium (3) in order to generate electrohydraulic shock waves. The electrodes (1) consist of a metallic work material. An electrical voltage is applied to the electrodes (1) in order to generate a voltage breakdown between the tips of the electrodes in the fluid medium (3), which work material consists of a tantalum alloy having a tantalum component greater than 60% or a tungsten alloy having a tungsten component greater than 60%.

### DEFINITIONS

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Extracorporeal” occurring or based outside the living body or plant structure.

A “generalized paraboloid” according to the present invention is also a three-dimensional bowl. In two dimensions (in Cartesian coordinates, x and y) the formula yⁿ=2px [with n being ≠2, but being greater than about 1,2 and smaller than 2, or greater than 2 but smaller than about 2,8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a defacto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude are below 100 ns. The duration of a shock wave is typically below 1-3 micro-seconds (μs) for the positive part of a cycle and typically above some micro-seconds for the negative part of a cycle.

‘Waves/wave fronts” described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 shows a schematic view of an apparatus for generating electrical discharge in a fluid medium for generating electrohydraulic shock waves with electrodes.

### DETAILED DESCRIPTION OF THE INVENTION

The task is solved in accordance with the generic part of Claim 1 in combination with its characterizing features starting from an apparatus for generating electrical discharge in a fluid medium for generating electrohydraulic shock waves. Various forms of such shock waves are explained in the definitions.

The task is solved in accordance with the invention in that an apparatus for generating electrical discharge in a fluid medium for generating electrohydraulic shock waves comprises electrodes consisting of a metallic work material in which fluid medium an electrical voltage is applied to the electrodes for the purpose of generating a voltage breakdown between the tips of the electrodes and which metallic work material consists of a tantalum alloy having a tantalum component greater than 60% preferably 80% or greater.

The solution has the advantage that electrodes are made available for the apparatus that have a very strong material with a low specific density.

In a further preferred embodiment the metallic work material of the electrodes 25 consists of a tantalum alloy with a wolfram component greater than 1%.

In a further preferred embodiment the metallic work material of the electrodes consists of a tantalum alloy with a titanium component greater than 1%.

In a further preferred embodiment the metallic work material of the electrodes consists of a tantalum alloy with an iron component greater than 1%.

In a further preferred embodiment the metallic work material of the electrodes consists of a tantalum alloy with a nickel component greater than 1%.

In a further preferred embodiment the metallic work material of the electrodes consists of the tantalum alloy TaW2.5% consisting of approximately 2.5% wolfram and 97.5% tantalum.

In a further preferred embodiment the metallic work material of the electrodes consists of a tantalum alloy with a hardness of at least 400 HV to 800 HV.

In a further preferred embodiment the metallic work material of the electrodes further comprises a surface layer of tantalum carbide or tungsten carbide which increases the electrodes' surface hardness so that it is close to the surface hardness of a diamond.

The solution has the advantage that electrodes are made available for the apparatus that have a very strong material with high conductivity, good corrosion resistance, high thermal resistance and a good mechanical workability quality that has a low specific density.

The invention is explained in detail in the following using the drawing.

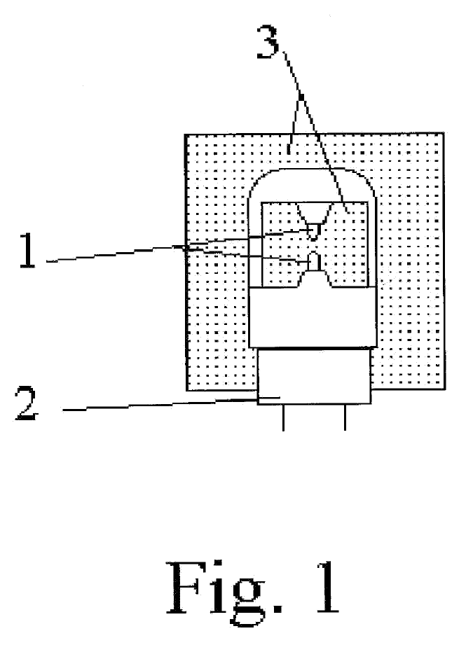

FIG. 1 shows a schematic view of an apparatus for generating electrical discharge in a fluid medium for generating electrohydraulic shock waves with electrodes.

FIG. 1 depicts an apparatus (2) for generating electrical discharge in a fluid medium (3) for generating electrohydraulic shock waves by means of electrodes (1).

In order to produce shock waves, the metallic electrodes (1) are used, between which a voltage breakdown takes place by the application of an electrical voltage. The electrical voltage causes a discharge that for its part generates a short, intensive shock wave in the fluid medium (3), e.g., water, which shock wave is used in medicine, e.g., for the removal of kidney stones.

The discharge of the electrical voltage in the fluid medium (3) has a destructive action on the metallic electrodes (1). The greater the material strength of the 5 electrodes (1) is, the less the destructive action of the discharges.

According to the invention a metallic work material with a very hard tantalum alloy is selected for the electrodes (1).

The solution has the advantage that electrodes (1) are made available for the apparatus (2) that have a very strong material with a low specific density.

In alternative embodiment the metallic work material can consist of a tungsten alloy component greater than 60% preferably 80% or greater.

The solution has the advantage that electrodes are made available for the apparatus that have a very strong material with a low specific density.

In a further preferred embodiment the metallic work material of the electrodes 25 consists of a tungsten alloy with a wolfram component greater than 1%.

In a further preferred embodiment the metallic work material of the electrodes consists of a tungsten alloy with a titanium component greater than 1%.

In a further preferred embodiment the metallic work material of the electrodes consists of a tungsten alloy with an iron component greater than 1%.

In a further preferred embodiment the metallic work material of the electrodes consists of a tungsten alloy with a nickel component greater than 1%.

In a further preferred embodiment the metallic work material of the electrodes consists of the tungsten alloy WTa2.5% consisting of approximately 2.5% tantalum and 97.5% tungsten.

In a further preferred embodiment the metallic work material of the electrodes consists of a tungsten alloy with a hardness of at least 400 HV to 800 HV.

In a further preferred embodiment the metallic work material of the electrodes further comprises a surface layer of tungsten carbide or tantalum carbide which increases the electrodes' surface hardness so that it is close to the surface hardness of a diamond.

The solution has the advantage that electrodes are made available for the apparatus that have a very strong material with high conductivity, good corrosion resistance, high thermal resistance and a good mechanical workability quality that has a low specific density.

The invention is explained in detail in the following using the drawing.

FIG. 1 shows a schematic view of an apparatus for generating electrical discharge in a fluid medium for generating electrohydraulic shock waves with electrodes.

FIG. 1 depicts an apparatus (2) for generating electrical discharge in a fluid medium (3) for generating electrohydraulic shock waves by means of electrodes (1).

In order to produce shock waves, the metallic electrodes (1) are used, between which a voltage breakdown takes place by the application of an electrical voltage. The electrical voltage causes a discharge that for its part generates a short, intensive shock wave in the fluid medium (3), e.g., water, which shock wave is used in medicine, e.g., for the removal of kidney stones.

The discharge of the electrical voltage in the fluid medium (3) has a destructive action on the metallic electrodes (1). The greater the material strength of the 5 electrodes (1) is, the less the destructive action of the discharges.

According to the invention a metallic work material with a very hard tungsten alloy is selected for the electrodes (1).

The solution has the advantage that electrodes (1) are made available for the apparatus (2) that have a very strong material with a low specific density.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

What is claimed is:

1. Apparatus (2) for generating electrical discharge in a fluid medium (3) for generating electrohydraulic shock waves, with electrodes (1) consisting of a metallic work material in which fluid medium (3) an electrical voltage can be applied to the electrodes (1) for the purpose of generating a voltage breakdown between the tips of the electrodes (1), characterized in that the metallic work material consists of a tantalum alloy having a tantalum component greater than 60%.

2. Apparatus according to claim 1, in which the tantalum alloy has an tungsten component greater than 1%.

3. Apparatus according to claim 1, in which the tantalum alloy has an niobium component greater than 1%.

4. Apparatus according to claim 1, in which the tantalum alloy has a titanium component greater than 1%.

5. Apparatus according to claim 1, in which the tantalum alloy has an iron component greater than 1%.

6. Apparatus according to claim 1, in which the tantalum alloy has a nickel component greater than 1%.

7. Apparatus according to claim 1, in which the tantalum alloy has a copper component greater than 1%.

8. Apparatus according to claim 1, in which the tantalum alloy has a hardness of at least 100 HV to 900 HV.

9. Apparatus according to claim 1, in which the tantalum alloy has a hardness of at least 400 HV to 600 HV.

10. Apparatus (2) for generating electrical discharge in a fluid medium (3) for generating electrohydraulic shock waves, with electrodes (1) consisting of a metallic work material in which fluid medium (3) an electrical voltage can be applied to the electrodes (1) for the purpose of generating a voltage breakdown between the tips of the electrodes (1), characterized in that the metallic work material consists of a tungsten alloy having a tungsten component greater than 60%.

11. Apparatus according to claim 10, in which the tungsten alloy has an tantalum component greater than 1%.

12. Apparatus according to claim 10, in which the tungsten alloy has an niobium component greater than 1%.

13. Apparatus according to claim 10, in which the tungsten alloy has a titanium component greater than 1%.

14. Apparatus according to claim 10, in which the tungsten alloy has an iron component greater than 1%.

15. Apparatus according to claim 10, in which the tungsten alloy has a nickel component greater than 1%.

16. Apparatus according to claim 10, in which the tungsten alloy has a copper component greater than 1%.

17. Apparatus according to claim 10, in which the tungsten alloy has a lanthanum component greater than 1%.

18. Apparatus according to claims 10 in which the tungsten alloy has a hardness of at least 100 HV to 900 HV.

18. Apparatus according to claims 10 in which the tungsten alloy has a hardness of at least 400 HV to 600 HV.
